(19) United States
(12) Patent Application Publication
(10) Pub. No.: US 2025/0082827 A1
(43) Pub. Date: Mar. 13, 2025

(54) TRILAYERED BIOMIMETIC HYDROGEL SCAFFOLDS OF DUAL MICROENVIRONMENT AND PREPARATION METHOD AND APPLICATION THEREOF

(71) Applicant: Xinxiang Medical College, Xinxiang (CN)

(72) Inventors: Yongkun Sun, Xinxiang (CN); Wenjie Ren, Xinxiang (CN); Zhengnan Zhang, Xinxiang (CN); Guangdong Zhou, Xinxiang (CN); Xianwei Wang, Xinxiang (CN); Xiaomeng Li, Xinxiang (CN); Yujie Hua, Xinxiang (CN); Shuhong Zhang, Xinxiang (CN); Zhikun Guo, Xinxiang (CN); Hongying Chen, Xinxiang (CN); Weiwei Zhao, Xinxiang (CN); Huanhuan Li, Xinxiang (CN); Zihan Zhao, Xinxiang (CN)

(21) Appl. No.: 18/959,494

(22) Filed: Nov. 25, 2024

(30) Foreign Application Priority Data
Dec. 1, 2023 (CN) 202311635480.4

(51) Int. Cl. A61L 27/52; A61L 27/26; A61L 27/36; A61L 27/54

(52) U.S. Cl. CPC A61L 27/52; A61L 27/26; A61L 27/3645; A61L 27/54; A61L 2430/02; A61L 2430/06

(57) ABSTRACT

The invention relates to a double-microenvironment three-layer bionic hydrogel scaffold and a preparation method and application thereof, which are characterized in that includes a cartilage layer GL-HPKGN, an intermediate layer GL-GMA, and a bone layer GL-HP/GMAAT; by an enzymatic crosslinking reaction based on hydroxyphenylpropionic acid (HPA), kartogenin (KGN) is grafted onto gelatin to form a cartilage-specific microenvironment in GL-HPKGN; Based on hydroxyphenylpropionic acid (HPA), a dual crosslinking network was formed by enzyme crosslinking reaction and methacryloyl-glycidyl dimethicone (GMA) photo-crosslinking reaction, which was used to graft atorvastatin (AT) onto gelatin, forming GL-HP/GMAAT with bone-specific microenvironment; the intermediate layer GL-GMA was beneficial for forming a clear and defined cartilage-bone integrated structure. The introduction of the tide-line intermediate layer GL-GMA facilitated the formation of well-defined chondro-bone integrated structures.

### TECHNICAL FIELD

The invention relates to the technical field of regenerative medicine, in particular to a three-layer biomimetic hydrogel scaffold having a mechanical and biological dual microenvironment, a preparation method and application thereof.

### BACKGROUND

Articular cartilage is an important tissue for joint movement and plays a vital role in bearing mechanical loads and reducing friction. Damage to articular cartilage, due to trauma, aging, degeneration and other reasons, inevitably extends to calcified cartilage and the subchondral bone layer, commonly known as osteochondral defects (OCDs). Under normal circumstances, articular cartilage has the physiological structure of cartilage, tidal line (calcified cartilage) and subchondral bone, and there are significant differences in mechanical properties and induced microenvironment. Among them, the biological function of the mesosphere tidal line is to maintain the constant interfacial structure and the dynamic balance of physiological calcification. The upper layer of cartilage is a relatively flexible part. The hyalinoid cartilage is mainly composed of type II collagen and proteoglycan. The active sites and spatial assembly of the two not only provide a suitable three-dimensional environment for the cells, but also give the cartilage sufficient elasticity and compressive strength. The lower bone, on the other hand, has a high hardness and serves as a mechanical support. The continuous development of tissue engineering technology has shown great potential and good application prospect in the regeneration and repair of cartilage and bone. Among them, the ideal hydrogel scaffold should have good biocompatibility, biodegradability, mechanical properties, cell adhesion, suitable scaffold aperture, degradation rate, easy to manufacture and other characteristics.

So far, scaffold materials used for chondro-bone repair still have the following problems: 1) Most studies use double-layer biomimetic scaffold to repair cartilage and subchondral bone, often ignoring the biomimetic construction of tidal line (calcified cartilage layer). At the same time, the gradient structure is a simple superposition of the upper and lower layers, and the connection between the adjacent layers is not close enough, prone to fracture, stratification and other phenomena, which cannot meet the requirements of the bionic construction of natural tissues; 2) Different levels of articular cartilage have different roles. The cartilage layer is relatively soft and wet to lubricate the joint cavity, the bone layer is hard to play the role of mechanical support, and the tide line firmly connects the soft and hard tissues to maintain the interface stability. 3) The biochemical microenvironment of scaffold material significantly affected the cartilage and bone differentiation of seed cells, and thus determined the therapeutic effect of tissue regeneration. Therefore, how to use bioactive materials to construct endogenous and differentiated microenvironment suitable for stem cell differentiation and activation (structural microenvironment, mechanical microenvironment, induced microenvironment) while biomimetic physiological structure to promote tissue regeneration is a basic scientific problem to be solved urgently in bone-cartilage tissue engineering.

### SUMMARY OF THE INVENTION

The invention designs a trilayered biomimetic hydrogel scaffolds of dual microenvironment and its preparation method and application. The technical problem solved by the scaffold is that there are usually significant differences in the physiological structure, mechanical function and biological microenvironment of joint osteochondral tissue. The biomimetic scaffold used for precise repair of joint osteochondral tissue includes: the development of cartilage layer, tidal line and subchondral bone layer is still elusive. The technical solution adopted by the present invention is as follows:

In order to solve the above-mentioned technical problems, the invention adopts the following schemes:

A Trilayered biomimetic hydrogel scaffolds of dual microenvironment, characterized in that: it includes a cartilage layer GL-HPKGN, an intermediate layer GL-GMA, and a bone layer GL-HP/GMAAT; by an enzymatic crosslinking reaction based on hydroxyphenylpropionic acid (HPA), kartogenin (KGN) is grafted onto gelatin to form a cartilage-specific microenvironment in GL-HPKGN; Based on hydroxyphenylpropionic acid (HPA), a dual crosslinking network was formed by enzyme crosslinking reaction and methacryloyl-glycidyl dimethicone (GMA) photo-crosslinking reaction, which was used to graft atorvastatin (AT) onto gelatin, forming GL-HP/GMAAT with bone-specific microenvironment; the intermediate layer GL-GMA was beneficial for forming a clear and defined cartilage-bone integrated structure.

Furthermore, The cartilage layer GL-HPKGN is prepared by adding gelatin GL to a mixture of hydroxyphenylpropionic acid (HPA), kartogenin (KGN), dimethyl sulfoxide (DMSO), ultrapure water, N-hydroxy succinimide, and 1-ethyl-3-(3-dimethylaminopropyl)-carbodiimide hydrochloride.

Furthermore, the gelatin-p-hydroxyphenylpropionic acid GL-HPAT is obtained by adding p-hydroxyphenylpropionic acid (HPA), atorvastatin (AT), dimethyl sulfoxide (DMSO), ultrapure water, N-hydroxysuccinimide, and 1-ethyl-3-(3-dimethylaminopropyl)-carbodiimide hydrochloride to gelatin GL.The bone layer GL-HP/GMAAT was prepared by mixing gelatin-p-hydroxyphenylpropionic acid GL-HPAT with methylpropenylglycide (GMA).

Furthermore, in the preparation of gelatin-p-hydroxyphenylpropionic acid GL-HP AT In the process, use HCl to gelatin-p-hydroxyphenylpropionic acid GL-HP AT The pH of the solution is adjusted to 4-5.

Furthermore, the intermediate layer GL-GMA is prepared by mixing gelatin GL with methyl propenyl glycidyl ester (GMA).

Furthermore, the cartilage layer GL-HPKGN was statically gelatinized under the enzymatic cross-linking reaction of horseradish peroxidase HRP and H2O2; Bone layer GL-HP/GMAAT was first gelatinized by enzyme cross-linking reaction of horseradish peroxidase HRP and H2O2, and then light curing was performed. The middle layer GL-GMA was quickly crosslinked to form hydrogel under light.

A method for preparing a trilayered biomimetic hydrogel scaffolds of dual microenvironment, including the following steps: by means of a dual crosslinking network based on hydroxyphenylpropionic acid (HPA) enzymatic crosslinking and methyl methacrylate (GMA) photo-crosslinking, atorvastatin (AT) is grafted onto gelatin to form GL-HP/GMAAT with bone-specific microenvironment.

A method for preparing a trilayered biomimetic hydrogel scaffolds of dual microenvironment, including the following steps: a dual crosslinking network based on enzymatic crosslinking of hydroxyphenylpropionic acid (HPA) and photo-crosslinking of methacryloyl glycerol (GMA) is used to graft atorvastatin (AT) onto gelatin, forming GL-HP/GMAAT with bone-specific microenvironment.

Application of a a trilayered biomimetic hydrogel scaffolds of dual microenvironment in the preparation of chondro-bone repair materials.

Application of a d a trilayered biomimetic hydrogel scaffolds of dual microenvironment in the repair of articular chondro-bone defects.

The present invention has the following beneficial effects:
(1) The invention grafts kartogenin (KGN) into gelatin through an enzyme cross-linking reaction based on p-hydroxyphenylpropionic acid (HPA) to form a cartilage specific microenvironment. The bone-specific microenvironment is achieved by grafting atorvastatin (AT) into gelatin as a subchondral bone layer through a double crosslinking network based on HPA enzyme crosslinking and a photocrosslinking reaction based on methyl acrylyl glycyl ester (GMA). The use of tidal lines as the intermediate layer for interfacial junctions is conducive to the formation of well-defined chondro-bone morphology and a better biomimetic chondro-bone integrated structure.
(2) The enzyme and optical double cross-linked hydrogel prepared by the invention is derived from gelatin, and is feasible in clinical operation for repairing cartilage defects, and has the characteristics of controllable shape and rapid prototyping, which is conducive to the repair of chondro-bone defects.
(3) The three-layer bionic hydrogel scaffold of the invention can successfully repair articular cartilage defect by activating endogenous repair, and provides a promising choice for future clinical treatment.

### DESCRIPTION OF THE DRAWINGS

FIG. 1 is the nuclear magnetic resonance spectrum of the double-microenvironment three-layer biomimetic hydrogel matrix of the invention.

FIG. 2 is the general picture of the three-layer biomimetic hydrogel formation of the double microenvironment of the invention.

FIG. 3 is the scanning electron microscope image of the freeze-dried scaffold of the three-layer bionic hydrogel of the double microenvironment.

FIG. 4 is the Live/Dead staining diagram of rabbit bone marrow mesenchymal stem cells loaded with the triple layer bionic hydrogel of the double microenvironment of the invention.

### DESCRIPTION OF EMBODIMENTS

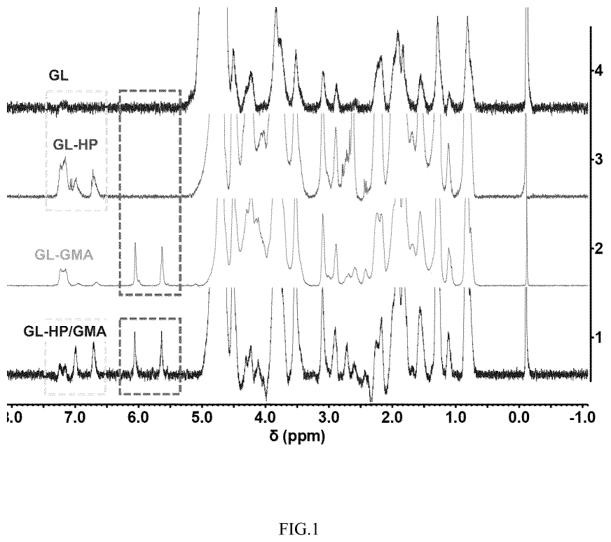

The invention is further explained in combination with FIGS. 1 to 4 below:

This invention provides a soft bone layer through an HPA-Based enzyme crosslinking reaction, KGN grafting to achieve a bone-specific microenvironment. The bone layer is achieved through a dual crosslinking network of an HPA-Based enzyme crosslinking reaction and a GMA-Based photo-crosslinking reaction, AT grafting to achieve a bone-specific microenvironment. Finally, a unified integration was achieved through three layers of biomimetic cartilage structures. The mechanical differences between the three layers were proved by in vitro experiments, where small molecule drugs were successfully grafted, and the scaffold exhibited good biocompatibility and chondrogenic effects. Meanwhile, in vivo experiments proved that by adding the three-layer biomimetic composite scaffold, bone-cartilage composite defect repair could be achieved.

### Embodiment 1

The double microenvironment three-layer bionic hydrogel preparation method comprises the following steps:

(1) For preparing the cartilage layer hydrogel: gelatine-p-hydroxyphenylpropionic acid (GL-HP).

1.32 g hydroxyphenylpropionic acid (HPA) was dissolved in 40 mL dimethyl sulfoxide (DMSO), After dissolution, 60 mL of Milli-Q water was added. 0.64 GN-hydroxysuccinimide and 0.76 b 1-ethyl-3-(3-dimethylaminopropyl)-carbonized diimide hydrochloride were dissolved in the above DMSO and water mixture, stirring at room temperature at high rotational speed to activate the carboxyl group. After 3 hours of agitation, 60 mL of 6.6% (w/v) gelatin solution was poured into the mixture and stirred at room temperature overnight. After the reaction is over, the above solution was transferred to a 14 kDa dialysis bag anddialyzed in Milli-Q water for 3 days. Finally freeze-dried GL-HP can be stored at-20° C.

In order to prepare GL-HPKGN, HPA and 10 mgKGN should be added in the above steps.

(2) The hydrogel gelatine-methylpropenyl glycidyl ester (GL-GMA) was prepared.

Stir 60 ml of 6.6% (w/v) gelatin solution at room temperature for half a day. After the reaction is over, the above solution was transferred to a 14 kDa dialysis bag and dialysis in Milli-Q water for 2-3 days. The reactants are lyophilized to obtain gelatin GL. After dissolving 2.5 gGL in milli-Q water (2%, w/v), adjust the solution pH to 4-5 with 1 M HCL. The prepared GL aqueous solution is added to 10 ml of methacrylglycol (GMA) at a rate of 0.5 ml/min. Reaction is at 50° C. for 24 hours, followed by dialysis in Milli-Q water at 40° C. for seven days using the above dialysis bag. After purification, it was freeze-dried and stored at −20° C. for subsequent use.

(3) Bone layer hydrogel GL-HP/GMA was prepared.

After the 2.5 gGL-HP macromer was dissolved in Milli-Q water (2%, w/v), the pH of the solution was adjusted to 4-5 with 1 M HCl. After this, the configured GL-HP aqueous solution was added with 10 ml methylpropenyl glycidyl ester (GMA) at a rate of 0.5 ml/min. The reaction was conducted at 50° C. for 24 h and the product was purified by dialysisagainst Milli-Q water with the above-mentioned dialysis membrane at40 'C for 7 days. After purification, it was freeze-dried and stored at −20° C. for subsequent use.

For the preparation of GL-HP/GMAAT, 60 mg of atorvastatin (AT) should be added to HPA in the first step of preparation of GL-HP.

### Embodiment 2

### NMR Detection of a Two-Microenvironment Three-Layer Biomimetic Hydrogel Matrix

All synthetic small molecules (GL-HP,GL-GMA,GL-HP/GMA) were confirmed by 1H NMR as follows. 1H NMR spectra were performed with a Varian INOVA spectrometer (Bruker, Billerica, MA, USA) with a uniaxial gradient inverse probe at a frequency of 300 MHz. Before measurement, 10 mg of the synthesized small molecule was dissolved in 1 mL of deuterium oxide containing 0.05% (w/v) 3-(trimethyl silver) propionate-2,2,3,3-D4 acid sodium salt (Sigma-Aldrich, St. Louis, Missouri, USA). Unfunctionalized raw gelatin was also tested as a control. This experiment was repeated three times independently. The hydrogel double bond grafting was identified, as shown in FIG. 1

### Embodiment 3

### Double Microenvironment Three-Layer Biomimetic Hydrogel Preparation and Gelation Diagram

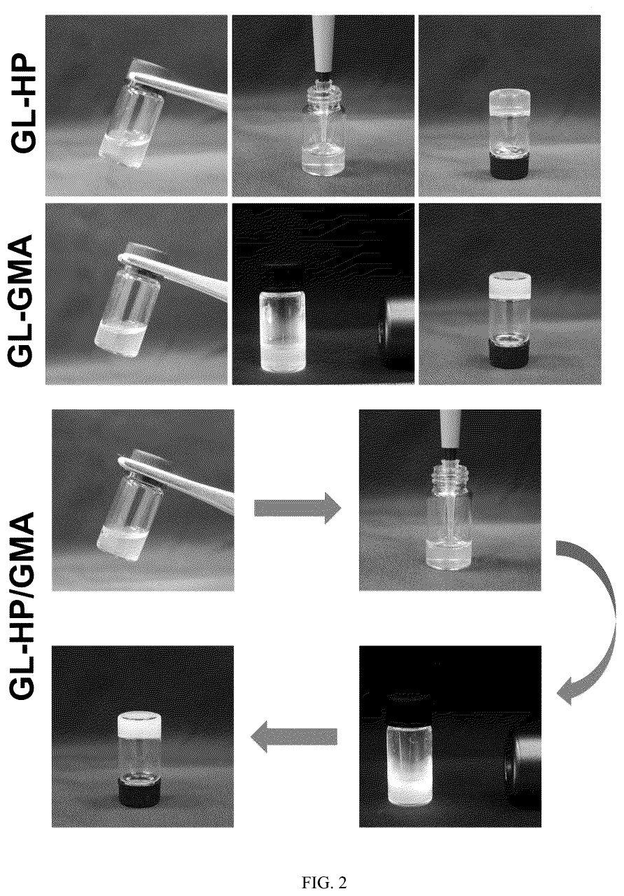

The crosslinking process (gelling: liquid-solid) is as follows: by preparing a solution of three layers of bionic hydrogel precursor (5% w/v-20% w/v) with a certain concentration, the cartilage layer GL-HP is statically gelling under the enzyme cross-linking reaction of horseradish peroxidase (HRP) and H2O2; The specific gumming process of GL-HP, GL-HP precursor solution (5% w/v-20%w/v), horseradish peroxidase (HRP) 0.15 units/ml, H2O2 0.85M/L, at room temperature static 20-30 s to gumming. The middle layer GL-GMA was quickly cross-linked to form hydrogel under light (405 nm); The bone layer GL-HP/GMA was first incubated into gel by enzyme cross-linking reaction of HRP and H2O2, and then cured by light, as shown in FIG. 2.

### Embodiment 4

### Freeze-Dried Scaffold with Three Layers of Bionic Hydrogel in Double Microenvironment Scanning Electron Microscope

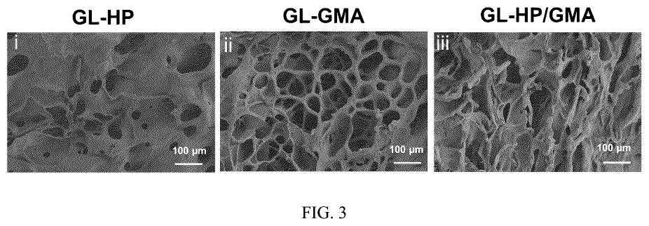

By preparing a three-layer bionic hydrogel precursor solution with a certain concentration (5% w/v-20% w/v), the cartilage layer GL-HP was statically gelled under the enzyme cross-linking reaction of horseradish peroxidase (HRP) and H2O2; The middle layer GL-GMA was quickly crosslinked to form hydrogel under light (405 nm); The bone layer GL-HP/GMA was first incubated into gel by enzyme-crosslinking reaction of HRP and H2O2, nd then cured by light. The material was freeze-dried in a refrigerator at −80° C. for 12 hours, and its microscopic morphology was observed under scanning electron microscope after gold spraying, as shown in FIG. 3.

### Embodiment 5

### Biocompatibility Analysis of Three-Layer Biomimetic Hydrogel in Double Microenvironment

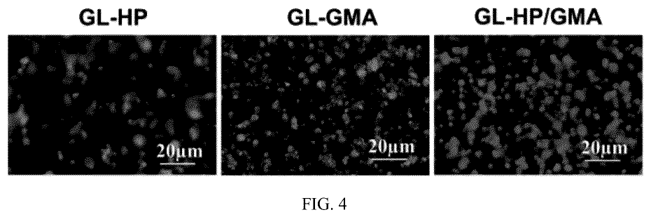

By preparing a certain concentration of two-microenvironment three-layer bionic hydrogel matrix precursor solution (5% w/v-20%w/v), using 0.22 μm filter to remove bacteria, The second-generation rabbit bone marrow mesymal stem cells were mixed evenly with the three-layer biomimetic hydrogel matrix precursor (GL-HP,GL-GMA,GL-HP/GMA) solution according to the cell volume of 5million/ml, and crosslinked to form hydrogel by the above method, and cultured in 24-well plates. 24 hours later, Calcein AM/PI staining was performed to analyze their biocompatibility, as shown in FIG. 4.

It is obvious that the realization of the invention is not limited by the above-mentioned methods. As long as various improvements of the method concept and technical scheme of the invention are adopted, or the idea and technical scheme of the invention are directly applied to other occasions without improvement, they are within the scope of protection of the invention.

What is claimed is:

1. A trilayered biomimetic hydrogel scaffolds of dual microenvironment, characterized in that: it includes a cartilage layer GL-HP_{KGN}, an intermediate layer GL-GMA, and a bone layer GL-HP/GMA_{AT}; By an enzymatic crosslinking reaction based on hydroxyphenylpropionic acid (HPA), kartogenin (KGN) is grafted onto gelatin to form a cartilage-specific microenvironment in GL-HP_{KGN}; Based on hydroxyphenylpropionic acid (HPA), a dual crosslinking network was formed by enzyme crosslinking reaction and methacryloyl-glycidyl dimethicone (GMA) photo-crosslinking reaction, which was used to graft atorvastatin (AT) onto gelatin, forming GL-HP/GMA_{AT} with bone-specific microenvironment; the intermediate layer GL-GMA was beneficial for forming a clear and defined cartilage-bone integrated structure.

2. The trilayered biomimetic hydrogel scaffolds of dual microenvironment according to claim 1, characterized in that:
the chondrogenic microenvironment-specific GL-HP_{KGN} layer is obtained by adding gelatin GL to a mixture of hydroxyphenylpropionic acid (HPA), kartogenin (KGN), dimethyl sulfoxide (DMSO), ultrapure water, N-hydroxysuccinimide, and 1-ethyl-3-(3-dimethylaminopropyl)-carbodiimide hydrochloride.

3. The trilayered biomimetic hydrogel scaffolds of dual microenvironment according to claim 2, characterized in that:
The gelatin-p-hydroxyphenylpropionic acid GL-HP_{AT} is obtained by adding p-hydroxyphenylpropionic acid (HPA), atorvastatin (AT), dimethyl sulfoxide (DMSO), ultrapure water, N-hydroxysuccinimide, and 1-ethyl-3-(3-dimethylaminopropyl)-carbodiimide hydrochloride to gelatin GL. The bone layer GL-HP/GMA_{AT} was prepared by mixing gelatin-p-hydroxyphenylpropionic acid GL-HP_{AT} with methylpropenylglycide (GMA).

4. The trilayered biomimetic hydrogel scaffolds of dual microenvironment according to claim 3, wherein:
the pH of the gelatin-p-hydroxyphenyl acetic acid (GL-HPA_{AT}) solution used in the preparation of gelatin-p-hydroxyphenyl acetic acid (GL-HPA_{AT}) is adjusted to 4-5 using HCl.

5. The trilayered biomimetic hydrogel scaffolds of dual microenvironment according to claim 1, wherein:
the intermediate layer (GL-GMA) is obtained by mixing gelatin (GL) with methyl methacrylate (GMA).

6. The trilayered biomimetic hydrogel scaffolds of dual microenvironment according to claim 2, wherein:
the cartilage layer (GL-HPKGN) is cross-linked into a gel by the enzymatic oxidation reaction between horseradish peroxidase (HRP) and H₂O₂;the bone layer (GL-HP/GMA_{AT}) is first cross-linked into a gel by the enzymatic oxidation reaction between horseradish peroxidase (HRP) and H₂O₂, and then subjected to light curing; the intermediate layer (GL-GMA) is quickly cross-linked into a hydrogel by light exposure.

7. The application of the trilayered biomimetic hydrogel scaffolds of dual microenvironment according to claim 1 in the preparation of cartilage-bone tissue regeneration materials.
